(19) United States
(12) Patent Application Publication
(10) Pub. No.: US 2026/0097375 A1
(43) Pub. Date: Apr. 9, 2026

(54) ACROLEIN AND/OR ACRYLIC ACID PRODUCTION FROM PROPYLENE WITH REDUCED AMOUNT OF HIGH BOILING POINT BYPRODUCTS

(71) Applicant: Rohm and Haas Company, Collegeville, PA (US)

(72) Inventor: Jinsuo Xu, Berwyn, PA (US)

(21) Appl. No.: 19/113,117

(22) PCT Filed: Sep. 26, 2023
(86) PCT No.: PCT/US2023/033648

§ 371 Date: Mar. 19, 2025

Related U.S. Application Data
(60) Provisional application No. 63/410,645, filed on Sep. 28, 2022.

(51) Int. Cl. B01J 8/02; B01J 23/00; B01J 35/00; C07C 45/35; C07C 51/25

(52) U.S. Cl. CPC B01J 8/0278; B01J 23/002; B01J 35/19; C07C 45/35; C07C 51/252; B01J 2208/025; B01J 2523/54; B01J 2523/68

(57) ABSTRACT

A reactor for producing acrolein and/or acrylic acid from propylene comprises an inlet for propylene to enter the reactor, an outlet for reaction products to exit the reactor, and a catalyst bed disposed between the inlet and the outlet. The catalyst bed comprises a molybdenum and bismuth based mixed oxide catalyst. The catalyst closest to the inlet of the reactor, CATᵢₙₗₑₜ, produces a lower amount of maleic anhydride to the total amount of acrolein and acrylic acid compared to the catalyst closest to the outlet of the reactor, CATₒᵤₜₗₑₜ.

### FIELD OF THE INVENTION

The present invention relates to a catalyst bed and process for the production of acrolein and/or acrylic acid from propylene.

### BACKGROUND OF THE INVENTION

Acrylic acid and its esters are important monomers for a variety of polymers including coatings, adhesives, and superabsorbent polymers. The majority of acrylic acid is produced via two-stage propylene oxidation, in which propylene is first oxidized over a molybdenum (Mo) and bismuth (Bi) based mixed oxide catalyst to primarily form acrolein with acrylic acid being a minor product. The acrolein is then oxidized over a molybdenum and vanadium (V) based mixed oxide catalyst to produce acrylic acid. Main byproducts of the acrylic acid production process from propylene include carbon monoxide, carbon dioxide, acetic acid, formaldehyde, acetaldehyde, propionic acid, propionaldehyde, acetone, allyl alcohol, maleic acid (or maleic anhydride), benzaldehyde, benzoic acid, furfural, and phthalic acids. These byproducts can be removed via distillation, extraction, melt crystallization, or combination of them. However, removal of these byproducts adds cost and complexity to the process.

U.S. Pat. No. 9,440,904 discloses a process for producing acrolein and/or acrylic acid, or methacrolein and/or methacrylic acid using two kinds of catalysts having different formulations that are stacked in an axial direction. In the two kinds of catalysts, the component amount of bismuth relative to molybdenum decreases from the gas inlet side toward the gas outlet side of the reactor, and the component amount of iron relative to molybdenum increases from the gas inlet side toward the gas outlet side.

There is need for a catalyst bed and process that is capable of reducing the amounts of byproducts formed when acrolein and/or acrylic acid is produced from propylene.

### SUMMARY OF THE INVENTION

The present invention is directed to a reactor and method for preparing acrolein and/or acrylic acid from propylene, and further, to produce acrylic acid from the acrolein.

According to one aspect of the present invention, a reactor for producing acrolein and/or acrylic acid from propylene comprises an inlet for propylene to enter the reactor, an outlet for reaction products to exit the reactor, and a catalyst bed disposed between the inlet and the outlet. The catalyst bed comprises a molybdenum and bismuth based mixed oxide catalyst. The catalyst closest to the inlet of the reactor, CATᵢₙₗₑₜ, produces a lower amount of maleic anhydride to the total amount of acrolein and acrylic acid compared to the catalyst closest to the outlet of the reactor, CATₒᵤₜₗₑₜ.

Another aspect of the present invention comprises a method for preparing acrolein and/or acrylic acid from propylene, the method comprising oxidizing propylene in a reactor comprising an inlet for propylene to enter the reactor, an outlet for reaction products to exit the reactor, and a catalyst bed disposed between the inlet and the outlet. The catalyst bed comprises a molybdenum and bismuth based mixed oxide catalyst. The catalyst closest to the inlet of the reactor, CATᵢₙₗₑₜ, produces a lower amount of maleic anhydride to the total amount of acrolein and acrylic acid compared to the catalyst closest to the outlet of the reactor, CATₒᵤₜₗₑₜ.

### DETAILED DESCRIPTION OF EMBODIMENTS OF THE INVENTION

As used herein, the terms “a,” “an,” “the,” “at least one,” and “one or more” are used interchangeably. The terms “comprises,” “includes,” “contains,” and variations thereof do not have a limiting meaning where these terms appear in the description and claims. Thus, for example, a mixture that includes a polymerization inhibitor can be interpreted to mean that the mixture comprises at least one polymerization inhibitor.

As used herein, recitations of numerical ranges by endpoints includes all numbers subsumed in that range (e.g. 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible subranges that are included in that range. For example, the range from 1 to 100 is intended to convey from 1.1 to 100, from 1 to 99.99, from 1.01 to 99.99, from 40 to 6, from 1 to 55, etc.

As used herein, the recitations of numerical ranges and/or numerical values, including such recitations in the claims, can be read to include the term “about.” In such instances, the term “about” refers to numerical ranges and/or numerical values that are substantially the same as those recited herein.

Unless stated to the contrary, or implicit from the context, all parts and percentages are based on weight and all test methods are current as of the filing date of this application. For purposes of United States patent practice, the contents of any referenced patent, patent application or publication are incorporated by reference in their entirety or its equivalent U.S. version is so incorporated by reference) especially with respect to the disclosure of definitions (to the extent not inconsistent with any definitions specifically provided in this disclosure) and general knowledge in the art.

One aspect of the present invention relates to a reactor for producing acrolein and/or acrylic acid from propylene.

The reactor comprises an inlet for propylene to enter the reactor, an outlet for reaction products to exit the reactor, and a catalyst bed disposed between the inlet and the outlet. The catalyst bed comprises a molybdenum and bismuth based mixed oxide catalyst.

As used herein, the term “reaction products” includes the desired products, including acrolein and/or acrylic acid, as well as by-products of the reaction, which may include carbon monoxide, carbon dioxide, acetic acid, formaldehyde, acetaldehyde, propionic acid, propionaldehyde, acetone, allyl alcohol, maleic acid, maleic anhydride, benzaldehyde, benzoic acid, furfural and phthalic acids.

Preferably, the molybdenum and bismuth based mixed oxide catalyst comprises a compound of Formula I:

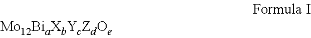

wherein:
X is at least one element selected from the group consisting of iron, cobalt, and nickel;
Y is at least one element selected from the group consisting of magnesium, calcium, strontium, barium, manganese, copper, zinc, cerium, boron, phosphorus, arsenic, tellurium, antimony, chromium, tungsten, sodium, potassium, rubidium, and cesium;
Z is at least one element selected from the group consisting of silicon, aluminum, titanium, tin, and zirconium;
a=0.1 to 2.0;
b=1 to 12;
c=0.01 to 3;
d=0 to 20; and
e is based on the oxidation states of the other elements in the catalyst.

One objective of the present invention is to minimize the formation of by-products. By-products of the reaction are typically removed via distillation, extraction, melt crystallization, or a combination thereof. The by-products not only consume valuable propylene feed, but also affect the recovery of acrylic acid and the need to remove the by-products to prepare certain grades of acrylic acid. For example, during distillation of crude acrylic acid to make overhead acrylic acid with reduced amounts of maleic anhydride or maleic acid, some acrylic acid must be left in the column bottom along with a majority of the maleic anhydride or maleic acid. For flocculant grade glacial acrylic acid, that limits the amount of furfural or benzaldehyde below 1 ppm, chemical scavengers such as m-phenylenediamine may need to be added to the crude acrylic acid to form higher boiling point adducts with high boiling point aldehydes that are removed in an additional step.

Without wishing to be bound by theory, it is believed that heavy byproducts are related to the formation of the intermediate 1,5-hexadiene. 1,5-Hexadiene is formed as an intermediate by-product when propylene is deprotonated to form a allyl radical that may then react with another allyl radical to form the 1,5-hexadiene. This reaction competes with the reaction of the allyl radical to form acrolein.

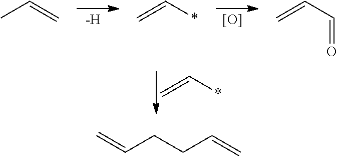

In the catalyst bed of the present invention, the molybdenum and bismuth based mixed oxide catalyst closest to the inlet of the reactor, CATᵢₙₗₑₜ, differs from the molybdenum and bismuth based mixed oxide catalyst closest to the outlet of the reactor, CATₒᵤₜₗₑₜ. CATᵢₙₗₑₜ produces a lower amount of maleic anhydride with respect to acrolein and acrylic acid compared to CATₒᵤₜₗₑₜ, where CATₒᵤₗₑₜ preferably has the same or higher activity than CATᵢₙₗₑₜ. This means that when measured independently of one another, CATᵢₙₗₑₜ produces a lower amount of maleic anhydride compared to the total amount of acrolein and acrylic acid in the reaction products under comparable propylene conversion than CATₒᵤₜₗₑₜ. As used herein, “comparable propylene conversion” means that the difference in the propylene conversion of CATᵢₙₗₑₜ and CATₒᵤₜₗₑₜ is less than 5%. Preferably, the propylene conversion difference between CATᵢₙₗₑₜ and CATₒᵤₜₗₑₜ is less than 3%, and more preferably less than 2%. This can be quantitatively measured by setting up two experiments that are identical with the exception of the catalyst and temperature and/or contact time to achieve a comparable propylene conversion, and measuring the concentration of maleic anhydride, acrolein, and acrylic acid in the reaction products, and calculating the ratio of maleic anhydride to the total amount of acrolein and acrylic acid according to the following equation:

$$Ratio [ \frac{M A N}{A c r + A A} ] = \frac{[ Moles of Maleic Anhydride ]}{[ Total Moles of Acrolein + Acrylic Acid ]}$$

To achieve a different ratio of maleic anhydride compared to the total amount of acrolein and acrylic acid in the reaction products between CATᵢₙₗₑₜ and CATₒᵤₜₗₑₜ, CATᵢₙₗₑₜ and CATₒᵤₜₗₑₜ may differ in at least one property or variable selected from composition, size, shape, method of preparation, carrier, concentration (e.g., amount of diluents), etc.

For example, the manner in which a catalyst is prepared may result in different catalytic activity. The preparation method may differ, for example, in order of addition of components (or elements), mixing temperatures and time, drying method, calcination temperatures and time, atmosphere, etc. Two catalysts having identical nominal compositions may have different catalytic activities and/or selectivities due to differences in the preparation method.

The composition of the catalyst may differ based on ratios of select elements (e.g., the molar ratio of bismuth to molybdenum), the addition or subtraction of elements, etc.

The structure of form of the catalyst or carrier may also alter the catalytic activity and/or selectivity. For example, a catalyst formed on spherical catalyst may have a different catalytic activity/selectivity compared to a catalyst with similar composition formed in a ring shape.

Preferably, CATᵢₙₗₑₜ and CATₒᵤₜₗₑₜ differ based on the composition of the catalyst.

The molybdenum and bismuth based mixed oxide catalyst of the catalyst bed may comprise a gradient composition in the catalyst from CATᵢₙₗₑₜ to CATₒᵤₜₗₑₜ. In the gradient composition, the catalyst closest to the inlet of the reactor, i.e., CATᵢₙₗₑₜ, may produce a ratio of maleic anhydride compared to the total amount of acrolein and acrylic acid that is lower than the catalyst closest to the outlet of the reactor, CATₒᵤₜₗₑₜ. The gradient composition may change linearly between the inlet and the outlet or may change non-linearly between the inlet and the outlet of the reactor. This gradient composition can be achieved, for example, by changing the relative amounts of components within the catalyst. For example, the molar ratio of bismuth to molybdenum may stay relatively constant near the inlet of the reactor and then rapidly increase towards the outlet. Alternatively, the gradient between CATᵢₙₗₑₜ and CATₒᵤₜₗₑₜ is a gradient in the composition of elements other than bismuth and molybdenum.

In another embodiment, the catalyst bed may comprise two or more zones within the molybdenum and bismuth based mixed oxide catalyst, wherein each zone produces a different ratio of maleic anhydride compared to the total amount of acrolein and acrylic acid in the reaction products. For example, the molar ratio of bismuth to molybdenum in the zone closest to the inlet, i.e., CATᵢₙₗₑₜ, may be lower than the molar ratio of bismuth to molybdenum in the zone closest to the outlet of the reactor, i.e., CATₒᵤₜₗₑₜ. Alternatively, each zone in the catalyst may have a different chemical formula, Mo₁₂BiₐX_{b}Y_{c}Z_{d}Oₑ.

The catalyst bed may comprise three or more zones, e.g., CAT₁ (where CAT₁=CATᵢₙₗₑₜ), CAT₂, and so on to CATₙ (where CATₙ=CATₒᵤₜₗₑₜ and n is the number of zones), within the molybdenum and bismuth based mixed oxide catalyst, wherein each zone comprises a catalyst that produces a different ratio of maleic anhydride compared to the total amount of acrolein and acrylic acid in the reaction products. When the catalyst bed comprises three or more zones within the molybdenum and bismuth based mixed oxide catalyst, the catalyst in the zone or zones, e.g., CAT₂ to CATₙ₋₁, between the zone closest to the inlet of the reactor (CAT₁ or CATᵢₙₗₑₜ) and the zone closest to the outlet of the reactor (CATₙ or CATₒᵤₗₑₜ) may produce a ratio of maleic anhydride to the total amount of acrolein and acrylic acid that is greater than the ratio of maleic anhydride to the total amount of acrolein and acrylic acid that is produced by the catalyst located in the zone closest to the inlet (CAT₁ or CATᵢₙₗₑₜ) and less than the ratio of maleic anhydride to the total amount of acrolein and acrylic acid that is produced by the catalyst in the zone located closest to the outlet of the reactor (CATₙ or CATₒᵤₜₗₑₜ).

Preferably, the catalyst bed comprises two or more zones within the molybdenum and bismuth based mixed oxide catalyst.

Preferably, the ratio of maleic anhydride to the total amount of acrolein and acrylic acid ranges from 250 to 10,000 ppm, where the ratio of maleic anhydride to the total amount of acrolein and acrylic acid is calculated by the formula shown above in terms of parts per million (ppm).

Preferably, the catalyst located closest to the inlet of the reactor produces maleic anhydride to the total amount of acrolein and acrylic acid in an amount ranging from 250 to 5,000 ppm, and more preferably from 250 to 4,000 ppm.

Preferably, the catalyst located closest to the outlet of the reactor produces maleic anhydride to the total amount of acrolein and acrylic acid in an amount ranging from 2,000 to 10,000 ppm, and more preferably from 2,000 to 8,000 ppm.

Another aspect of the present invention relates to a method for preparing acrolein from propylene comprising oxidizing propylene in a reactor comprising an inlet for introducing propylene to the reactor, an outlet for reaction products to exit the reactor, and a catalyst bed disposed between the inlet and the outlet of the reactor. The catalyst bed comprises a molybdenum and bismuth based mixed oxide catalyst, as described above, wherein the catalyst located closest to the inlet of the reactor, CATᵢₙₗₑₜ, has a lower ratio of maleic anhydride compared to the total amount of acrolein and acrylic acid in the reaction products compared to the catalyst located closest to the outlet of the reactor, CATₒᵤₜₗₑₜ. The method is performed in the gas phase in the presence of oxygen.

In a second step, the acrolein in the reaction products may be selectively oxidized over a second mixed metal oxide catalyst in the presence of oxygen in the vapor phase, wherein the second mixed metal oxide catalyst has a different composition from the molybdenum and bismuth based mixed oxide catalyst.

The second mixed metal oxide catalyst may be, for example, a solid catalyst that comprises oxides of molybdenum (Mo) and vanadium (V). The second mixed metal oxide catalyst may also contain at least one additional element selected from tungsten (W), copper (Cu), iron (Fe), antimony (Sb), and phosphorus (P). When the second mixed metal oxide catalyst contains at least one additional element, the molybdenum and vanadium are the main metal elements present. Preferably, the second mixed metal oxide catalyst comprises at least 40 wt. % of molybdenum and vanadium based on the total weight of metals in the second mixed metal oxide catalyst, such as, for example, at least 50 wt. %, at least 60 wt. %, or at least 70 wt. %.

The second mixed metal oxide catalyst can be any commercially available mixed metal oxide catalyst used for oxidation of acrolein to acrylic acid.

In the selective oxidation reaction to form acrolein and/or acrylic acid, the oxygen can be present in the form of purified oxygen, oxygen in air, or lattice oxygen of the mixed metal oxide catalyst. Preferably, the oxygen is from air or the lattice oxygen of the mixed metal oxide catalyst.

Purification of the acrolein and/or acrylic acid can be achieved by one or more techniques known in the art, such as, for example, absorption using water or an organic solvent, extraction, fractional distillation, or melt crystallization.

### Prophetic Example

The following prophetic examples illustrates the present invention but is not intended to limit the scope of the invention.

In a tubular reactor, a catalyst bed will be placed between an inlet of the reactor and an outlet of the reactor. The catalyst bed will be composed of a molybdenum and bismuth based mixed oxide catalyst comprising two zones, where the first zone is approximately one third of the volume of the catalyst bed and the second zone is approximately two thirds of the volume of the catalyst bed. The catalysts will be provided from different sources and have differing maleic anhydride to acrolein/acrylic acid production ratios by virtue of having different compositions and produced by different methods. The catalyst producing the lower maleic anhydride to total acrolein/acrylic acid production ratio will have a ratio of about 2500 ppm. The catalyst producing the higher maleic anhydride to total acrolein/acrylic acid production ratio will have a ratio of about 4300 ppm at a similar conversion rate, i.e., within 2%, as the first catalyst.

In a first experiment according to the present invention, the catalyst having the lower maleic anhydride to total acrolein/acrylic acid production ratio will be located in the first zone nearest the inlet and the catalyst having the higher maleic anhydride to total acrolein/acrylic acid production ratio will be located in the second zone nearest the outlet.

In a comparative second experiment, the catalyst having the higher maleic anhydride to acrolein/acrylic acid production ratio will be located in the first zone nearest the inlet and the catalyst having the lower maleic anhydride to acrolein/acrylic acid production ratio will be located in the second zone nearest the outlet.

A reactant gas stream comprising 8.1 vol % propylene and 14.4 vol % oxygen will be introduced to the inlet of the reactor. To obtain similar conversions, the temperatures in the reactor will be adjusted so that the overall conversion for each experiment is within 2% of each other.

The first experiment is expected to produce significantly less maleic anhydride than the second experiment.

1. A reactor for producing acrolein and/or acrylic acid from propylene, the reactor comprising an inlet for propylene to enter the reactor, an outlet for reaction products to exit the reactor, and a catalyst bed disposed between the inlet and the outlet, wherein the catalyst bed comprises a molybdenum and bismuth based mixed oxide catalyst, wherein the catalyst closest to the inlet of the reactor, CATinlet, produces a lower amount of maleic anhydride compared to the total amount of acrolein and acrylic acid compared to the catalyst closest to the outlet of the reactor, CAToutlet.

2. The reactor of claim 1, wherein a ratio of maleic anhydride to a total amount of acrolein and acrylic acid for CATinlet is less than a ratio of maleic anhydride to a total amount of acrolein and acrylic acid for CAToutlet.

3. The reactor of claim 1, wherein CATinlet differs from CAToutlet in at least one property or variable selected from composition, size, method of preparation, carrier, and concentration.

4. The reactor according to claim 1, wherein the catalyst bed comprises a gradient composition, wherein the composition of the catalyst changes from CATinlet to CAToutlet.

5. The reactor according to claim 1, wherein the catalyst bed comprises two or more zones, wherein the catalyst located in the first zone comprises CATinlet, and the catalyst in the second zone comprises CAToutlet.

6. The reactor according to claim 5, wherein the catalyst bed comprises three or more zones, wherein the catalyst in the zone or zones located between the zone closest to the inlet and the zone closest to the outlet produces a ratio of maleic anhydride to the total amount of acrolein and acrylic acid that is greater than the ratio of maleic anhydride to the total amount of acrolein and acrylic acid produced by catalyst located in the zone closest to the inlet, CATinlet, and less than the ratio of maleic anhydride to the total amount of acrolein and acrylic acid produced by the catalyst in the zone located closest to the outlet of the reactor, CAToutlet.

7. The reactor according to claim 1, wherein the molybdenum and bismuth based mixed oxide catalyst comprises a compound of Formula I:
Mo₁₂BiₐX_{b}Y_{c}Z_{d}Oₑ Formula I wherein:
X is at least one element selected from the group consisting of iron, cobalt, and nickel;
Y is at least one element selected from the group consisting of magnesium, calcium, strontium, barium, manganese, copper, zinc, cerium, boron, phosphorus, arsenic, tellurium, antimony, chromium, tungsten, sodium, potassium, rubidium, and cesium;
a=0.1 to 2.0;
b=1 to 12;
c=0.01 to 3;
d=0 to 20; and
e is based on the oxidation states of the other elements in the catalyst.

8. The reactor according to claim 1, wherein the molybdenum and bismuth based mixed oxide catalyst produces a ratio of maleic anhydride to the total amount of acrolein and acrylic acid ranging from 250 to 10,000 ppm.

9. The reactor according to claim 1, wherein CATᵢₙₗₑₜ produces a ratio of maleic anhydride to a total amount of acrolein and acrylic acid ranging from 250 to 5,000 ppm, and CATₒᵤₜₗₑₜ produces a ratio of maleic anhydride to a total amount of acrolein and acrylic acid ranging from 2,000 to 10,000 ppm.

10. The reactor of claim 9, wherein CATᵢₙₗₑₜ produces a ratio of maleic anhydride to a total amount of acrolein and acrylic acid ranging from 250 to 4,000 ppm, and CATₒᵤₜₗₑₜ produces a ratio of maleic anhydride to a total amount of acrolein and acrylic acid ranging from 2,000 to 8,000.

11. A method comprising:
oxidizing propylene in a reactor according to claim 1, wherein the reaction products exiting the reactor comprise acrolein.

12. The method according to claim 11, further comprising oxidizing the acrolein to produce acrylic acid.
